(19) United States
(12) Patent Application Publication
(10) Pub. No.: US 2021/0393486 A1
(43) Pub. Date: Dec. 23, 2021

(54) CONTROLLED COMPLIANCE MEDICATION DISPENSER SYSTEM

(71) Applicants: Shayon Choudhury, Cincinnati, OH (US); Rishi Gabbita, Mason, OH (US)

(72) Inventors: Shayon Choudhury, Cincinnati, OH (US); Rishi Gabbita, Mason, OH (US)

(21) Appl. No.: 17/355,695

(22) Filed: Jun. 23, 2021

Related U.S. Application Data
(60) Provisional application No. 63/042,880, filed on Jun. 23, 2020.

(51) Int. Cl. A61J 7/00; G16H 10/60; G16H 20/13

(52) U.S. Cl. CPC A61J 7/0076; G16H 10/60; A61J 2200/30; A61J 2200/74; G16H 20/13

(57) ABSTRACT

A system for permitting administration of an as-needed medication dose to a patient is disclosed herein. The system comprises a medication management server, a computing device associated with a healthcare provider and a dispenser associated with a patient. The dispenser in communication with the server is configured to dispense controlled medication at a minimum dosing interval. The dispenser comprises a controller in communication with the server configured to control the actuation of one or more events of the dispenser including a medication dispensation event. The computing device in communication with the server is configured to manage a quantity of the medication dose dispensed and the predefined dosing interval, remotely. The server is further configured to send an alert on tampering the dispenser or on detecting an out-of-communication state of dispenser for a predetermined time interval.

### CROSS-REFERENCE TO RELATED APPLICATIONS

This application claims the benefit of priority under 35 U.S.C. § 119(e) to U.S. Patent Application Provisional Application Ser. No. 63/042,880, entitled “CONTROLLED COMPLIANCE MEDICATION DISPENSER SYSTEM”, filed Jun. 23, 2021, the contents of which are hereby incorporated by reference in their entirety for any purpose.

### BACKGROUND

1. Technical Field

The present disclosure relates generally to a medication dispenser, and more particularly, to a system for permitting administration of an as-needed medication dose to a patient at a predetermined dosing interval.

2. Description of the Related Art

Numerous medical conditions and illnesses require adherence of treatment regimens that often include precisely scheduled drug administration over a specific and often lengthy time period. Variance from the regimen or failure of compliance can diminish the therapeutic effectiveness of the regimen and lead to delayed recovery or create complications in treatment, which not only prolongs a patient's suffering but also increases the cost of associated care. In some cases, patients often simply forget to take their medicine, and complications are sometimes brought on by patients who miss one or more pills and then attempt to “catch up” by taking more than the prescribed dosage. The difficulty in remembering when to take prescribed medication is greater when a patient is prescribed drugs of different types required to be taken at different times.

Elderly patients frequently do not have sufficient mental alertness to keep track of the frequencies and dosages of their various medicines over a sustained period of time. Such patients also frequently suffer from impaired manual dexterity, which makes handling of individual tablets and capsules a difficult task, which some patients consequently avoid to their detriment. In other cases, especially concerning pain management, compliance failure can lead to drug addiction by over-medication. In still other cases, a patient can take the prescribed amount of medication but outside of the prescribed schedule, leading to the patient's being under- or over-medicated at different times.

Further, the practitioners/health care provider should assume that all patients are potentially non-compliant, and work to individualize recommendations specific to the different needs of different patients. During regular check-ups, the practitioner should ask the patient about the latter's compliance between the previous visit and the current visit. Finally, it should be noted that a patient's actual behavior may differ from their reported behavior. In short, patients may provide inaccurate reports of their compliance. A variety of products and techniques for reminding patients during medication regimens are known and are generally cost prohibitive.

Therefore, there is a need for a system for permitting administration of an as-needed medication dose to a patient at a predetermined dosing interval. Further, there is a need for a system that allows a healthcare provider to track patient medication compliance, adjust medication dose, and predefined dosing interval, remotely.

### BRIEF SUMMARY

The present invention discloses a system for permitting administration of an as-needed medication dose to a patient at a predetermined dosing interval.

The dispenser in communication with the server is configured to dispense controlled medication at a minimum dosing interval. The dispenser comprises a controller in communication with the server configured to control actuation of one or more events of the dispenser including a medication dispensation event. The computing device configured to allow the user to manage and customize a quantity of the medication dose dispensed and the predefined dosing interval. The server comprises one or more program modules to track time interval for drug accessibility. The server controls the controller of the dispenser to provide access to single dose of medication after each minimum time interval.

The server is further configured to monitor and record one or more events performed by the dispenser. The server is further configured to provide a chart showing trends and changes in the administration of the as-needed medication dose and the predetermined dosing interval. The server is further configured to send an alert on detecting abnormal events of the dispenser including tampering of the dispenser and an out-of-communication state of dispenser for a predetermined time interval. The server is further configured to predict and propose a dosage regimen or change in dosing interval based on the recorded events of the dispenser. The server is further configured to provide patient medication compliance history calculated through records of medication dispensing events.

As will be described in detail below, an intelligent medication dispenser is disclosed that provides automated dispensing of medication for personal use by a patient. The intelligent medication dispenser disclosed herein enables programming of medication information and medication policy information for a patient via a network interface, rather than a simple time of day mechanism to dispense medication. The intelligent medication dispenser disclosed herein enables bulk refilling of one or more medications simultaneously. The intelligent medication dispenser disclosed herein has the ability to automatically recognize individual medications upon refilling as well as upon dispensing. The intelligent medication dispenser disclosed herein is enabled to internally store and track a plurality of individual medications and to dispense each medication individually for the patient. The intelligent medication dispenser disclosed herein enables a patient to be in direct communication with a caregiver for support or assistance. The intelligent medication dispenser disclosed herein provides for packaging and labeling of certain doses of medications to enable the patient to travel and still maintain adherence to prescriptions. The intelligent medication dispenser disclosed herein is enabled to provide notifications and alerts to caregivers, physicians, and to patients (users) for tracking medication usage, as well as aberrations or errors. The intelligent medication dispenser disclosed herein is enabled to securely authenticate the user before dispensing medication.

The above summary contains simplifications, generalizations and omissions of detail and is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed written description.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIG. 1 exemplarily illustrates an environment of a system for permitting administration of an as-needed medication dose to a patient at a predetermined dosing interval, according to an embodiment of the present invention.

FIG. 2 exemplarily illustrates a block diagram of control components of the system, according to an embodiment of the present invention.

### DETAILED DESCRIPTION

The present invention discloses a system for permitting administration of an as-needed medication dose to a patient at a predetermined dosing interval. The system further allows a healthcare provider to track patient medication compliance, adjust medication dose and predefined dosing interval, remotely.

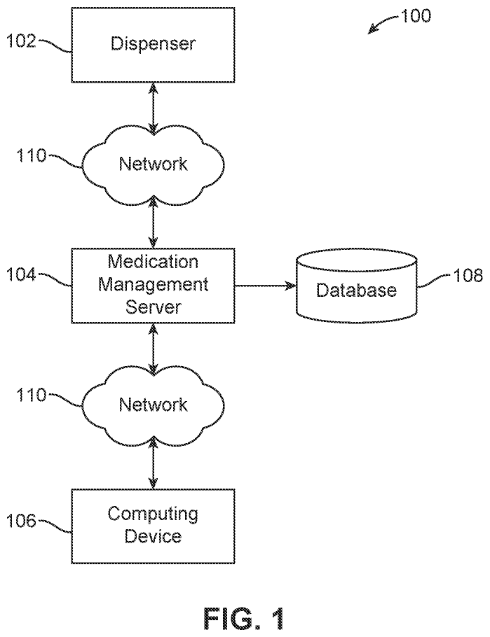

FIG. 1 discloses an environment 100 of a system for permitting administration of an as-needed medication dose to a patient at a predetermined dosing interval. The environment 100 includes a medication management server 104, a computing device 106 associated with a healthcare provider, and a dispenser 102 associated with a patient. More specifically, the dispenser 102 is a device that patient possesses, and dispenser 102 may either be a portable device or a mounted device present in the location where the patient resides.

In accordance with an embodiment of the present disclosure, the computing device 106 and the dispenser 102 are in communication with the medication management server 104 via a network 110. Network 110 generally represents one or more interconnected networks, over which the computing device 106 and server 104 can communicate with each other. Network 110 may include packet-based wide area networks (such as the Internet), local area networks (LAN), private networks, wireless networks, satellite networks, cellular networks, paging networks, and the like. A person skilled in the art will recognize that network 110 may also be a combination of more than one type of network. For example, network 110 may be a combination of a LAN and the Internet. In addition, network 110 may be implemented as a wired network, or a wireless network or a combination thereof.

In an embodiment, the at least one database 108 may be accessible by the medication management server 104. In another embodiment, the at least one database 108 may be integrated into the medication management server 104 or separate from it. In an embodiment, the at least one database 108 resides in a connected server or in a cloud computing service. In an embodiment, regardless of location, the at least one database 108 comprises a memory to store and organize certain data for use by the medication management server 104. In one embodiment, the at least one database 108 stores profile data, diagnosis data of the patient, records of one or more events performed by the dispenser 102, and the like. In one embodiment, the profile data comprises email addresses, data identifying the patient, image of the patient, and location of the patient.

In one embodiment, the server 104 is at least one of a general or a special purpose computer. In an embodiment, it operates as a single computer, which can be a hardware and/or software server, a workstation, a desktop, a mainframe, a supercomputer, a server farm, and so forth. In an embodiment, the server 104 is in communication with network 110. Such communication can be via a software application, a mobile app, a browser, an OS, and/or any combination thereof.

In an embodiment, the computing device 106 could be a touchscreen and/or a non-touchscreen device operable on any type of OS, such as iOS™, Windows™, Android™, Unix™ Linux™ and/or others. The computing device 106 could be any type of electronic device configured to operate as at least one of a desktop, a laptop, a tablet, a mobile phone, and mobile and/or handheld electronic devices. The computing device 106 includes an operating system that coordinates the use of hardware and software resources, as well as one or more applications (e.g., web browser, web application) that perform specialized tasks for the service provider. The computing device 106 includes functionality to communicate, obtain and/or execute instructions or applications using the network 110. Such communication can be via a software application, a mobile app, a browser, an OS, and/or any combination thereof.

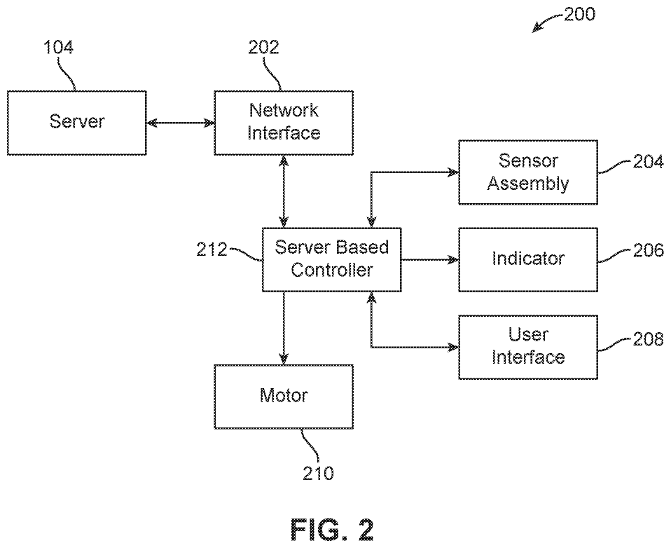

The dispenser 102, in communication with the server 104, is configured to dispense controlled medication at a minimum dosing interval. The dispenser 102 comprises a controller 212, shown in FIG. 2, in communication with the server 104 and configured to control the actuation of one or more events of the dispenser 102 including a medication dispensation event. The computing device 106 is configured to allow the user to manage and customize a quantity of the medication dose that is to be dispensed along with the predefined dosing interval.

The controller 212 may include, but is not limited to, a central processing unit (CPU), a memory, a storage unit, and a transceiver, thereto. The CPU may perform all calculation and processing functions. The CPU may interact with the memory by running/executing programs and/or retrieving data from the storage unit. The storage unit may maintain a database and/or storage of a user's biometric information, whether the user is an authorized user, a time of when the user may access the medication as authorized, and any third party information. The controller 212 may be physically and electrically connected to a biometric scanner, at least one battery, a recharge port, a sensor, a timer, a light, and a lock. As such, controller 212 may control and/or regulate all the functions of all the components within dispenser 102. Additionally, a timer may provide functionality, such as a clock, a countdown, and/or an alarm. The transceiver may allow communication between the controller 212 and an external control unit, which may occur with a wired and/or wireless connection using BLUETOOTH, near-field communication (NFC), WIFI, satellite, RFID communication, etc. As such, the transceiver 121d may allow the dispenser 102 to be tracked via a GPS signal.

Memory may include, in general, any computer-readable medium (also referred to as a processor-readable medium) that may include any non-transitory (e.g., tangible) medium that participates in providing medication information or instructions that may be read by a computer. Such a medium may take many forms, including, but not limited to, non-volatile media and volatile media. Non-volatile media may include, for example, optical or magnetic disks and other persistent memory. Volatile media may include, for example, dynamic random access memory (DRAM), which typically constitutes a main memory. Such instructions may be transmitted by one or more transmission media, including radio waves, metal wire, fiber optics, and the like, including the wires that comprise a system bus coupled to a processor of a computer. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, or any other medium from which a computer can read.

Further, databases, data repositories or other medication information stores described herein may generally include various kinds of mechanisms for storing, providing, accessing, and retrieving various kinds of medication information, including a hierarchical database, a set of files in a file system, an application database in a proprietary format, a relational database management system (RDBMS), etc. Each such medication information store may generally be included within (e.g., memory) or external (e.g., database or cloud storage) to a computing system and/or device (e.g., dispenser 102) employing a computer operating system such as one of those mentioned above, and/or accessed via a network or connection in any one or more of a variety of manners. A file system may be accessible from a computer operating system, and may include files stored in various formats. An RDBMS generally employs the Structured Query Language (SQL) in addition to a language for creating, storing, editing, and executing stored procedures, such as the PL/SQL language mentioned above. Memory and database may be connected to or part of any portion of system.

The external control unit may be a device having wireless communication capabilities, including, but not limited to, a cellular telephone, a laptop computer, a desktop computer, a tablet computer, a PDA, a smart watch, etc. Furthermore, the external control unit may control and/or regulate all the functions of all the components within the cap, and may allow the CPU to be programmed to include information relating to the user and/or third party information. Also, the external control unit may display a map thereon to correspond to the GPS signal emitted by the transceiver, in order to track and monitor a location of the dispenser 102.

The biometric scanner may include at least one of a fingerprint scanner, an iris scanner, a facial scanner, a voice scanner (collectively, biometric information), etc., but is not limited thereto. The storage unit may be initialized with the user's biometric information via at least one of a third party entering data into the storage unit using previously obtained biometric information or an initial biometric scan using the biometric scanner upon the user obtaining the dispenser 102.

Further, in some embodiments, the programmable medication dispenser may further include a tampering sensor configured to detect at least one tampering action performed on the programmable medication dispenser. Further, the tampering sensor may be configured to detect the breaking of at least one compartment of the plurality of compartments, including but not limited to, the storage compartment and the battery compartment.

Further, the tampering sensor may be communicatively coupled to the processing device. Further, the processing device may be configured to generate at least one tampering alert based on detection of the at least one tampering action. Further, the communication device may be configured to transmit the at least one tampering alert to at least one user device.

Further, in some embodiments, the programmable medication dispenser may further include at least one chemical container and at least one chemical actuator. Further, the at least one chemical container may include a chemical agent.

Further, the at least one chemical actuator coupled to the at least one chemical container. Further, the at least one chemical actuator may be configured to release the chemical agent into at least one compartment of the plurality of compartments when the at least one chemical actuator may be activated. Further, the at least one chemical actuator may be communicatively coupled to the tampering sensor. Further, detection of the at least one tampering action causes activation of the at least one chemical actuator. Further, the chemical agent may be configured to react with a medication contained in the at least one compartment in order to render the medication unusable.

Further, in some embodiments, the programmable medication dispenser may further include at least one chemical container and at least one chemical actuator. Further, the at least one chemical container may include a chemical agent.

Further, the at least one chemical actuator coupled to the at least one chemical container. Further, the at least one chemical actuator may be configured to release the chemical agent into at least one compartment of the plurality of compartments when the at least one chemical actuator may be activated. Further, the at least one chemical actuator may be communicatively coupled to the processing device. Further, the processing device may be further configured to generate a chemical release command. Further, receipt of the chemical release command causes activation of the at least one chemical actuator.

Further, the programmable medication dispenser may further include at least one positioning system. Further, the at least one positioning system may be communicatively coupled with the communication device. Further, the at least one positioning system may be configured to determine geographical location data of the programmable medication dispenser. Further, the communication device may be configured to transmit the geographical location data to the at least one user device.

Further, in an embodiment, the housing may include a tamper-proof material. Further, the tamper-proof material may include at least one of stainless steel, a metal alloy, a high strength acrylic laminate, and a composite plastic. Further, in an embodiment, the at least one bottomless tray may include of at least one of chemical-resistant plastic, chemical-resistant acrylic, and chemical-resistant stainless steel. Further, in an embodiment, the at least one compartment wall of the plurality of compartments may be coated with at least one of an abrasive and a vitreous material configured to perform at least one of abrade and deflect a drill-bit.

In one or more embodiments, the dispenser 102 may be configured to receive, store, monitor, display and transfer medication information. The dispenser 102 may be configured for monitoring the dosage and usage of prescription medication. The dispenser 102 may also be configured to dispense the prescription medication according to required dosages.

Medication information may include any information associated with medication or medicine. Medication information may be associated with or include a prescription (e.g., patient name, prescriber name, strength, dosage, quantity, expiration, use directions, or drug or diet interactions), a container type (e.g., shape, size, or color), patient information (e.g., name or history), or a combination thereof. Medication information may include other information associated with a patient or medication. Prescription information may include medication information associated with a patient or as prescribed or defined by a user such as medical professional or a patient.

In one or more embodiments, a patient authorization process is included that comprises the entry of numeric or alphabetic characters via a keypad or other identification techniques for determining if the patient is authorized to receive the medication. For example, a fingerprint or voice print identification could be used. In another embodiment, a bar code reader can be incorporated into the dispenser 102 for reading a bar code assigned to the patient and printed on the patient's identification, such as a wristband. If the stored bar code in the reader matches the scanned bar code and the minimum dosing interval has elapsed, the dispenser 102 can then provide patient access to the next medication dose.

The server 104 is further configured to monitor and record one or more events performed by the dispenser 102. The server 104 is configured to predict and propose a dosage regimen or change in dosing interval based on the activity of the dispenser 102 recorded over time. In another embodiment, the physician/healthcare provider can change the dosing interval based on the activity of the dispenser 102 recorded over time, via the computing device 106.

For example, a patient could be taking a medication, e.g., an opioid, and then the patient could be prescribed a co-therapy, e.g., a nutritional supplement, which may include zinc, GABA, calcium, magnesium, B-vitamins, antioxidants, omega-3s, probiotics, and the like. By monitoring the time intervals between the dosages over time, the physician can gain insight into which dietary supplements help aid opiate addiction recovery, wherein the insights may include medication composition, dosage regimen, combinations, etc. In another aspect, the time intervals can be altered in real time based on the patient's response. In one exemplary embodiment, the dosage interval may be increased or decreased remotely.

In one embodiment, the server 104 may be configured to provide a visual depiction illustrating trends and changes in the administration of the as-needed medication dose and the predetermined dosing interval. In one embodiment, the server 104 may further be configured to send an alert on detecting abnormal events associated with the dispenser 102, including tampering of the dispenser 102 and an out-of-communication state of dispenser 102 for a predetermined time interval. In yet another embodiment, the server 104 may also be configured to compute and provide patient medication compliance history by using the records of medication dispensing events associated with the dispenser 102 occurring during a particular time interval.

Although the description herein generally refers to the healthcare provider as the party exercising control over the operation of the dispenser 102, this function can be performed by any third party ultimately controlling the patient's medication dosing, such as an in-home care giver, a medical technician, a pharmacy staff member, a physician, and the like.

FIG. 2 exemplarily illustrates a block diagram 200 of the control components of the system, according to an embodiment of the present invention. In accordance with one embodiment, the dispenser 102 may include a plurality of medication compartments and a receptacle configured to receive medications from the plurality of compartments. The dispenser 102 may further include a door for providing access to the receptacle. The door may be configured to move between an open position and a closed position, through the action of a motor 210 and associated gearings.

In accordance with an exemplary implementation of the present system, once the patient has opened and closed the door to retrieve a medication dosage, a timing sequence may be initiated and during that sequence, the medication receptacle may be locked in place. After the interval has elapsed, the server-based controller 212 may be configured to control and dispense the medication to the receptacle. The controller 212 may further transmit signals to operate the motor 210 and associated gearings to move the door to open position and provide access to the medication receptacle.

In another embodiment, the dispenser 102 may further include a sensor assembly 204 for determining a quantity of medication in the receptacle or the plurality of compartments. In yet another embodiment, the dispenser 102 may also include an indicator 206 for indicating the remaining quantity of medication, events related to dosing interval, elapse of dosing interval or time of consuming the medication, and other similar events or information.

In one embodiment, the sensor assembly 204 comprises a weight sensor. In another embodiment, the dispenser 102 uses light impedance to count the pills/medication. The dispenser 102 further comprises a battery assembly to supply power to the components of the dispenser 102. The battery assembly has a capacity to power the dispenser 102 for at least two to three months. The dispenser 102 further comprises a user interface 208 to control the device manually.

In another embodiment, the dispenser 102 may be configured to either AC and or DC power, such as a lithium ion battery system. If the dispenser 102 uses rechargeable batteries, the power source may monitor the power remaining and give reminder signals to recharge batteries once the batteries are low. In another embodiment, the dispenser 102 may further comprise

[symbol]

In another embodiment, the dispenser 102 may further include memory, which may store medication information including prescription information, medicine container identification information and patient's medical history information. In another embodiment, the dispenser 102 may further include an I/O device including, but is not limited to, an RF reader, one or more cameras, scanner, barcode reader, one or more lights, one or more sensors, one or more speakers, one or more buttons, keyboard, mouse, touch screen, microphone, one or more scales, or a combination thereof.

The server 104 comprises one or more program modules to track time interval for drug accessibility. The server 104 communicates with the dispenser 102 via a network interface 202. The server 104 controls the controller 212 of the dispenser 102 to provide access to single dose of medication after each minimum time interval. The activities or events of the dispenser 102 are continuously monitored and stored at the database 108 of the server 104. The server 104 is further configured to compute and provide visual depictions indicating the trends and changes associated with a patient's progress while the patient is undertaking the dosage regimen using the present system over a time period corresponding to the treatment period. The server 104 is further configured to send an alert to the relevant authority on detecting abnormal events of the dispenser 102 including tampering of the dispenser 102 and an out-of-communication state of dispenser 102 for a predetermined time interval. The relevant authority may be a physician or healthcare worker associated with the patient, in accordance with one embodiment.

The server 104 is configured to change the time frame according to a schedule, for example, 6 hours minimum wait until the next dose for each dose, during week 1; 7 hours minimum wait until the next dose for each dose, during week 2; 8 hours minimum wait until the next dose for each dose, during week 3; 12 hours minimum wait until the next dose for each dose, during week 4. For example, if a patient can only have 1/dose per 6 hours, they have to wait 6 hours to get the next dose. The patient receiving the dose resets the time to zero so that the patient has to wait a minimum 6 hours until the next dose. The patient prevented to access a missed dose from the dispenser 102.

The server-controlled controller 212 is configured to dispense only a certain number of pills per hour or day and keep the patients from indiscriminate use of the drug. The server 104 is further configured to verify the medication compliance exhibited by the patient and report the same to the healthcare provider. In accordance with one embodiment, the healthcare provider may check patient's compliance and change the dosage and the predetermined dosage interval, remotely. The system limits the number of visits to the doctor for changing the dosage. In another embodiment, the server 104 is configured to check the patient's compliance and change the dosage and the predetermined dosage interval. The server 104 is further configured to continuously monitor the dispenser 102 to prevent tampering of the dispenser 102. If the dispenser 102 is out of communication for a predefined time interval, the server 104 is configured to send an alert to the healthcare provider regarding the state of the dispenser 102.

An advantageous aspect of the present invention is that the present invention reduces the expenditure associated with visits to the doctor visits. Another advantageous aspect is that the present invention facilitates storage and dispensation of a particular medicine or medicines for a prolonged period of time, e.g., 3 months or more. Yet another advantageous aspect of the present invention is that it provides patient compliance history though records of dispensing sent to server 104, thereby allowing a healthcare provider to learn the patient's behavior and change the dosage intervals and dosages accordingly, wherein such a change may be remotely operated. One more advantageous aspect of the present invention is that it locks the leftover medication/missed medication in the dispenser 102, thereby eliminating the chances of indiscriminate consumption of the medication.

The present disclosure includes many aspects and features. Moreover, while many aspects and features relate to, and are described in the context of a programmable medication dispenser, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor, and at least one actuator. Examples of the one or more client devices and/or the server computer may include, a desktop computer, a laptop computer, a tablet computer, a personal digital assistant, a portable electronic device, a wearable computer, a smartphone, an Internet of Things (IoT) device, a smart electrical appliance, a video game console, a rack server, a super-computer, a mainframe computer, mini-computer, micro-computer, a storage server, an application server (e.g., a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice-based interface, gesture-based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third-party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role-based access control, and so on.

Further, one or more steps of the method disclosed herein may be initiated, maintained, controlled and/or terminated based on a control input received from one or more devices operated by one or more users such as, for example, but not limited to, an end-user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human-readable secret data (e.g. username, password, passphrase, PIN, secret question, secret answer etc.) and/or possession of a machine-readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human-readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular system, device or component thereof to the teachings of the disclosure without departing from the essential scope thereof. Therefore, it is intended that the disclosure not be limited to the particular embodiments disclosed for carrying out this disclosure, but that the disclosure will include all embodiments falling within the scope of the appended claims. Moreover, the use of the terms first, second, etc. do not denote any order or importance, but rather the terms first, second, etc. are used to distinguish one element from another.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

What is claimed is:

1. A system for permitting administration of an as-needed medication dose to a patient at a predetermined dosing interval, comprising:
a medication management server;
a dispenser in communication with the server configured to dispense controlled medication at a minimum dosing interval, wherein the dispenser comprises a controller in communication with the server configured to control actuation of one or more events of the dispenser including a medication dispensation event; and
a computing device in communication with the server configured to manage and customize a quantity of the medication dose dispensed and the predefined dosing interval;
wherein the server is configured to:
send instructions to the controller to control the actuation of one or more events of the dispenser including the medication dispensation event;
monitor and record one or more events performed by the dispenser;
provide one or more visual depictions showing trends and changes in the administration of the as-needed medication dose and the predetermined dosing interval;
facilitate management and customization of a quantity of the medication dose and the predefined dosing interval;
predict and propose a dosage regimen or change in dosing interval based on the recorded events of the dispenser; and
send an alert on detecting abnormal events of the dispenser including tampering of the dispenser and an out-of-communication state of dispenser for a predetermined time interval.

2. The medication dispensing system according to claim 1, wherein the computing device and the dispenser are in communication with the server via a network.

3. The medication dispensing system according to claim 1, further comprises at least one database in communication with the dispenser configured to store information related to the events of the dispenser and information related to healthcare providers and patients.

4. The medication dispensing system according to claim 1, wherein the one or more events include displaying a number of dispensed dosage and a total number of dosages remaining in the dispenser.

5. The medication dispensing system according to claim 1, wherein the dispenser further comprises an indicator in electronic communication with the controller is configured to display a number of dispensed dosage and a total number of dosages remaining in the dispenser.

6. The medication dispensing system according to claim 1, wherein the dispenser further comprises a sensor assembly to determine a quantity of medication dose.

7. The medication dispensing system according to claim 1, wherein the dispenser further comprises a weight sensor to determine the quantity of medication dose.

8. The medication dispensing system according to claim 1, wherein the server is configured to provide patient medication compliance history calculated through records of medication dispensing events.
